(19) United States
(12) Patent Application Publication
(10) Pub. No.: US 2004/0077998 A1
(43) Pub. Date: Apr. 22, 2004

(54) Intravenous tubing covering

(76) Inventor: Nancy J. Morris, Mishawaka, IN (US)

Correspondence Address: BAKER & DANIELS, 205 W. JEFFERSON BOULEVARD, SUITE 250, SOUTH BEND, IN, 46601, US

(21) Appl. No.: 10/600,859

(22) Filed: Jun. 20, 2003

Related U.S. Application Data
(60) Provisional application No. 60/390,869, filed on Jun. 21, 2002.

(51) Int. Cl. A61M 31/00

(52) U.S. Cl. 604/93.01; 604/171

(57) ABSTRACT

An intravenous tubing line cover includes an enclosure for covering the transparent components of an intravenous infusion apparatus to shield light-sensitive fluids and medications. The cover is constructed from an opaque fabric such as flannel, polyester, or cotton-blend material and includes covering sections to accommodate an IV bag and tubing lines of various lengths. Connecting straps are included to interconnect tubing line cover sections around infusion pumps and roller clamps. The covers are reusable being provided with fasteners for attachment around the various transparent components of the IV apparatus.

### BACKGROUND OF THE INVENTION

This application claims the benefit of U.S. Provisional Patent Application Serial No. 60/390,869 filed Jun. 21, 2002, the complete disclosure of which is hereby expressly incorporated by reference.

The invention relates to a covering for intravenous tubing used in the delivery of medications or fluids to a patient.

It is common in the medical field to administer medications and other fluids to patients by intravenous infusion. This is particularly common in neonatal and premature infant care where infants may not be able to nurse effectively or where their gastrointestinal tracts may not be sufficiently developed to digest feedings. It is also common in critically ill infants and children who cannot be fed orally because of their disease process. In intravenous infusion, an infusion pump or syringe pump is used to administer a prescribed amount of medication or fluids over a certain period of time. Infusion products are typically delivered to the patient in a clear, see-through plastic IV (intravenous) bag and dispensed through clear tubing.

The problem associated with this dispensing method is that certain fluids are light sensitive, such as in the administration of intralipids which supply essential fatty acids to patients needing intravenous nutrition. Another problem area involves hyperalimentation where nutrients including vitamins and minerals, electrolytes, and certain drugs such as insulin are administered intravenously. When exposed to light, intralipids can turn into peroxides; and, in the case of hyperalimentation, light exposure produces oxygen-free radicals. Both of these light-sensitive changes have been associated with at least three diseases in the neonatal wards, Chronic Lung Disease (CLD), Necrotizing Enterocolitis (NEC), Retinopathy of Prematurity (ROP). It is known to use light-sensitive tubing, to shield light-sensitive IV fluids. However, this tubing is expensive and must be replaced on a daily basis making it costly to use.

Furthermore, particularly in neonatal wards, parents of hospitalized children, when the children are hospitalized for long periods of time, have commented about the harsh nature of the hospital surroundings, with IV tubing and numerous pumps and apparatus connected to their infants. Thus it is desirable to provide a system which not only prevents the issues as discussed above, but also provides a softer approach for the care unit facility. One covering is shown in U.S. Pat. No. 6,315,759 for covering intravenous lines, but is comprised of a closed cell foam and is used as a protective cover for some of the intravenous lines, and therefore does not solve the problems as described above.

These and other disadvantages are addressed by the subject of the present application.

### SUMMARY OF THE INVENTION

These and other objects of the invention are accomplished by providing a reusable intravenous tubing covering system that in one embodiment comprises a cover for an IV bag and sleeve-type covers for multiple tubing sections from the IV bag to an infusion control pump to the patient. Connecting straps are provided to interconnect tubing line covers at the IV pump and at roller clamps.

In another embodiment, the intravenous tubing covering system is adapted for use with a syringe pump infusion apparatus and includes a tubing sleeve covering the length of the IV tubing line exiting the syringe pump. A strap is provided for securing the tubing cover to the syringe pump unit.

The IV bag cover and each of the tubing cover sections are constructed from an opaque fabric material such as flannel, polyester, or cotton blends. The tubing cover sections are constructed from fabric pieces that are wrapped around the tubing and joined with fasteners along the length of the cover.

### BRIEF DESCRIPTION OF THE DRAWINGS

FIG. 1 is a perspective view of an IV apparatus including a bag cover and an IV tubing cover according to the present invention;

FIG. 2 is a perspective view of a syringe pump apparatus including a tubing cover according to the present invention;

FIG. 2A is diagrammatic view of an embodiment showing both of the pumps of FIGS. 1 and 2 joined together;

FIG. 3 is a view showing an IV tubing cover according to the FIG. 2A embodiment in use with IV infusion and syringe pumps;

FIG. 4 is a view showing the syringe pump and associated tubing line cover of FIG. 3 in more detail; and

FIG. 5 is a view showing the IV pump and adjacent sections of tubing line cover of FIG. 3 in more detail.

### DESCRIPTION OF THE PREFERRED EMBODIMENT

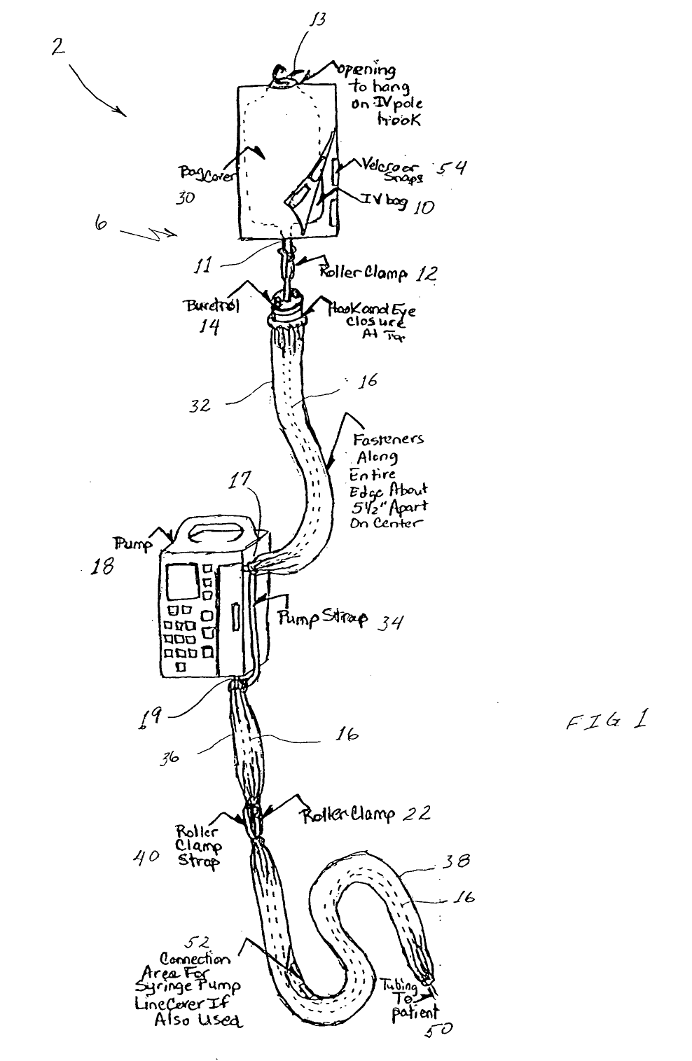

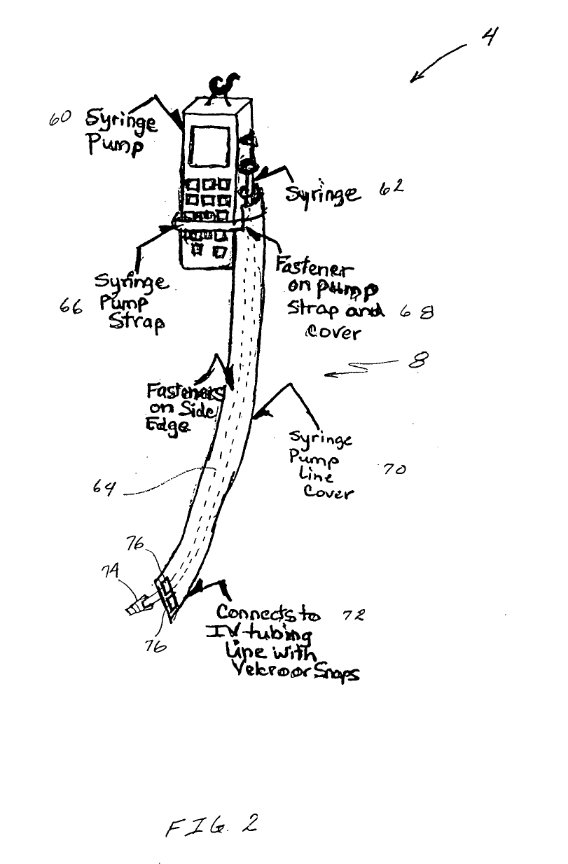

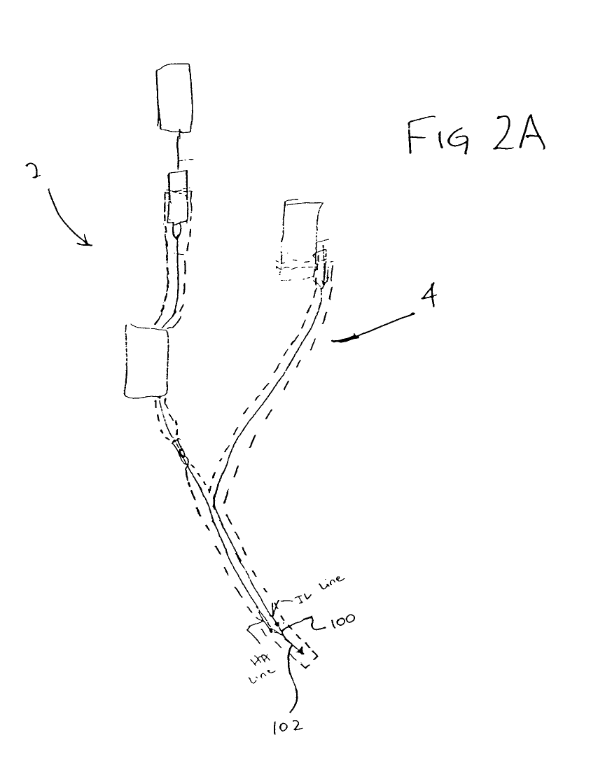

With reference to FIGS. 1 and 2 therein shown respectively, an intravenous (IV) infusion apparatus 2 and a syringe pump apparatus 4, both employing a tubing cover system according to the present invention. The IV arrangement of FIG. 1 is typical of the type used in hyperalimentation. The apparatus includes an IV bag 10 containing the fluid that is to be infused, and an infusion control pump 18 that is programmed to administer the fluid at a specified rate for a specified period of time. An IV bag outlet tube 11 delivers the IV fluid to a buretrol 14. An upper roller clamp 12 is provided on the outlet tube prior to the buretrol 14 for opening and closing the IV bag outlet tube 11. An IV tubing line 16 begins at the buretrol 14 and is routed to the infusion pump 18. The IV tubing line 16 exits the pump and continues on to the patient. A lower roller clamp 22 is present on the tubing line between the infusion pump and the patient so that fluid flow to the patient can be stopped if necessary.

With reference still to FIG. 1, the transparent components of the IV apparatus are shown with a cover system 6 according to the present invention to protect light-sensitive fluids. An IV bag cover 30 encloses the IV bag 10. The IV bag cover includes a layer of opaque fabric material of sufficient size to completely cover the IV bag 10. The bag cover can be fashioned from two pieces of fabric sewn together. With the typical small IV bag, fabric pieces sized about 7 inches in width and 12 inches in length (7″×12″) would be recommended. For the typical large IV bag, fabric pieces sized 7 inches by 16 inches would be recommended. However, other dimensions are also contemplated as would be appropriate for nonstandard-sized bags. Alternatively, the bag cover could be made from a single fabric piece folded to produce a covering of similar dimensions. The fabric pieces can be assembled to make the bag cover by sewing the pieces together leaving sufficient space unsewn for insertion and removal of the IV bag. The portions not permanently sewn can be joined by fasteners 54 so that the IV bag can be inserted and the cover then sealed. The fasteners can be a VELCRO hook and loop material (VELCRO is the registered trademark of Velcro Industries B.V. of The Netherlands), snaps, or any other suitable fasteners. Alternatively, fasteners can be applied to the entire perimeter of the fabric instead of sewing the fabric. With either method, the IV bag cover must be provided with an opening along the top edge to provide access to the tab on the IV bag so that the IV bag can be suspended from an IV pole hook 13.

The tubing line cover 32 shields the IV tubing from the buretrol 14 to the pump 18. In a typical infusion setup, the length of this portion of tubing line 16 connecting the buretrol 14 to the IV pump 18 is approximately 36 inches. This dimension, of course, can be varied. The cover for this portion of line 16 is preferably constructed from a single fabric piece that is essentially rectangular in shape and having fasteners (not shown) along its length. The cover is formed by wrapping the fabric around the buretrol 14 and the tubing line 16 and sealing the opposing lengthwise edges with the fasteners. One end of the cover is sealed around the buretrol 14 and the other is sealed around the tubing line 16 at the pump inlet 17 using any suitable means. For instance, the fabric could have an elastic band sewn into each end or could have a fold-over hook and loop type fastener. Alternatively, an external elastic band or a draw string tie could be applied to seal each end of the cover.

Tubing line cover 36 covers the portion of the tubing line 16 between the IV pump 18 and the lower roller clamp 22. Cover 36 is similar in construction to tubing line cover 32 except that it is shorter in length. As with the other tubing sections, the length of this section is also variable.

Tubing line cover 38 covers the portion of the tubing line 16 from the lower roller clamp 22 to the end of the catheter tubes at 50. Tubing line cover 38 is also similar in construction to tubing line cover 32; however, in addition to being longer than tubing line cover 32, tubing cover 38 also is provided with extra fasteners 52 so that the cover can be sealed about an interconnecting IV line such as from a syringe pump. The fasteners can be varied or be the entire length of the tubing line cores to allow for more than one interconnecting syringe pump line covers to be connected.

With continued reference to FIG. 1, it is shown that at the IV pump 18, IV tubing line 16 exits the cover 32, and enters the IV pump 18 at the inlet 17, then exits IV pump 18 and enters tubing cover 36. A strap section 34 is provided to join cover sections 32 and 36 and has sufficient length to span the distance between the inlet 17 and outlet 19 of IV pump 18. Similarly, a second strap 40 is provided to join tubing sections 36 and 38. Strap section 40 has sufficient length to span the roller clamp 22. Preferably, strap sections 34 and 40 contain an elastic insert extending the length of each strap section.

With reference now to FIG. 2, the IV tubing cover of the present invention is shown with syringe pump arrangement 4 as might be used for the infusion of intralipids or medications. The syringe pump apparatus 4 includes a syringe pump unit 60 that controls administration of the fluids or medication, syringe 62, and transparent tubing line 64. As depicted in FIG. 2, the tubing line is equipped with a fitting 74 commonly used for connection to another IV line. This tubing line may also be equipped with a bifuse that unites two separate lines into one line. Another option shown in FIG. 2A, where the syringe tubing line 64 runs into the IV tubing cover at the connection site and lie in the IV tubing line cover with the IV tubing Line 16 and a bifuse 100, placing the tubing lines closer to the patient. The combined line 102 would be directed to the patient's peripheral IV, umbilical line, or PICC line.

The cover system 8 includes a tubing line cover 70 that extends the length of tubing line 64 which is typically about 37 inches. This dimension however is freely variable. The tubing cover 70 is preferably constructed from a fabric piece about 7 inches in width and about 37 inches in length, with the length of the fabric being commensurate with the length of the tubing to be covered. Fasteners (not shown) such as VELCRO®) hook and loop material or snaps are spaced along the fabric length. The cover 70 is formed by wrapping the fabric around the tubing line 64 and joining the fasteners.

A strap 66 is affixed to the upper end of cover 70 for securing the cover 70 around the syringe pump unit 60. Preferably, the strap 66 includes an elastic insert to secure the cover 70 to the pump unit 60. Alternatively, VELCRO® hook and loop material or snap fasteners or any other suitable tying device could be used. At its lower end 72, the cover 70 includes an extra VELCRO® fastener 76 for connecting the cover 70 to an IV tubing line cover such as that shown in FIG. 1 and previously described.

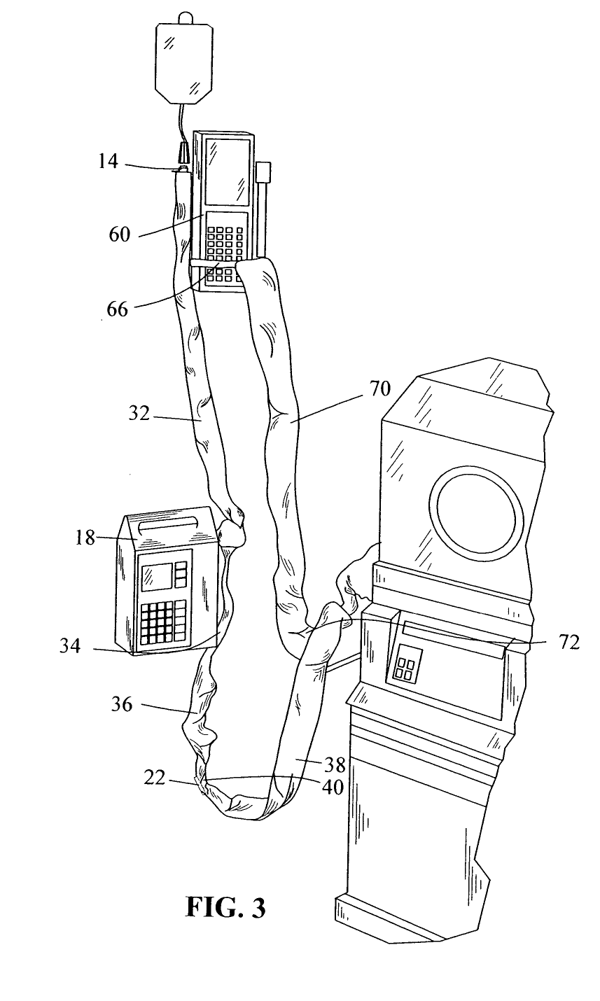

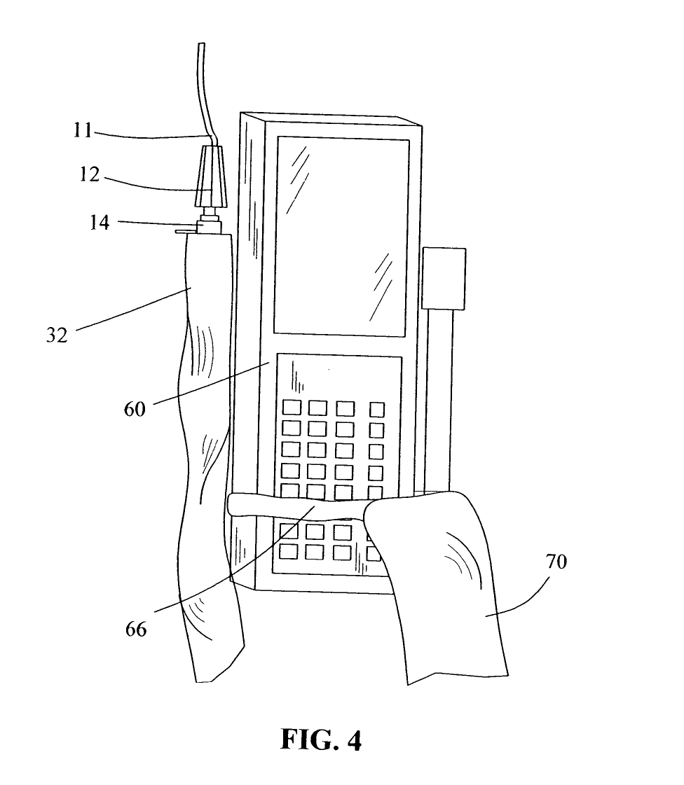

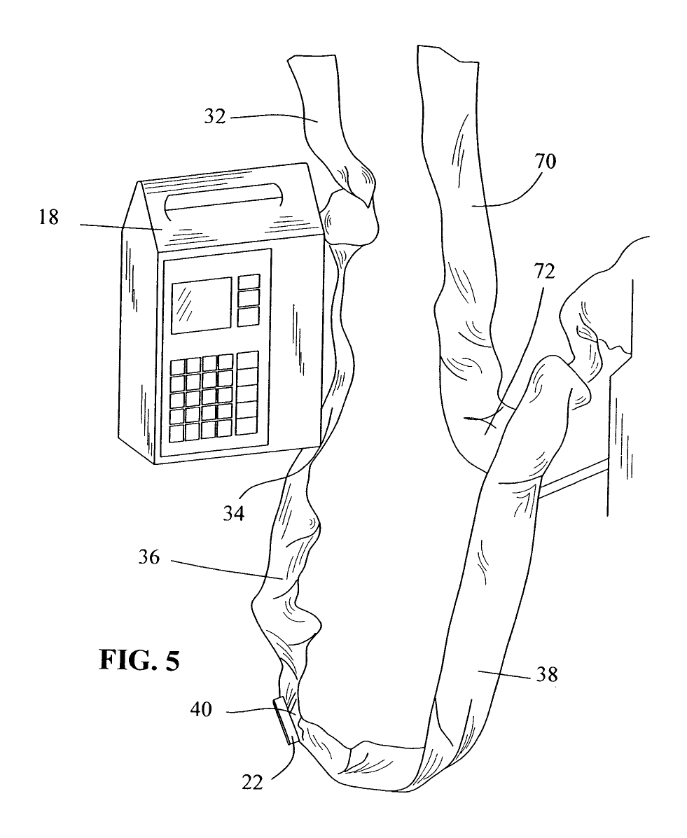

With reference now to FIGS. 3-5, one embodiment of an IV tubing line cover is shown attached to an infusion apparatus in a hospital setting. In FIG. 3, a covered IV pump system is shown in combination with a covered syringe pump system with the syringe pump line interconnected to the IV pump line. This arrangement could be used for the combined administration of fluids and medications. The end of the syringe pump tubing line cover 72 attaches to cover 38 at the juncture of the IV pump tubing line and the syringe pump tubing line. In FIG. 4, the syringe pump and associated tubing line cover and pump strap are shown in more detail with the application of strap 66 to the syringe pump 60 clearly demonstrated. In FIG. 5, the IV pump and associated tubing line cover are shown in more detail with the interconnection of cover sections 32, 36, and 38 by strap portions 34 and 40 clearly demonstrated.

A preferred embodiment of the intravenous tubing covering hereinbefore described may be constructed as follows:

Bag Cover

Small: 2 7″×12″ fabric pieces

2 fasteners (snaps or VELCRO®) material)

Large: 2 7″×16″ fabric pieces

2 fasteners

Sew edge of fabric pieces together at sides and top edge, leaving three inches in the center of top edge for a hanger tab opening. Sew one side and half of the other side. Turn over ½″ on edges and finish (by hemming or fancy stitching). Leave bottom edge open and hem. Place fasteners on the open edge of the side that was half sewn.

IV Tubing Line Cover

From Buretrol cover to pump

One 7″×36″ fabric piece

One 4″ piece of ¼″ elastic

One 2″ piece of ¼″ elastic

One hook and eye

6 fasteners

All side edges are the longer edges. The shorter edges at the top and bottom are for casings.

The cover can be assembled by the following process: Turn over side edges ½″ and finish, and sew ½″ casing at top edge. Insert the 4″ piece of elastic, and sew at each end. Sew ½″ casing at the bottom edge, and insert 2″ piece of elastic, and sew at each end. Sew fasteners about every 5 ½″ to close side edge. A continuous fastener may be applied if preferred.

Pump Strap

One 1 ½″×12″ fabric piece

One 10″ piece of ¼″ elastic

Fold right side in, sew ⅛″ lengthwise seam. Turn right side out. Insert elastic and sew both ends. Sew one end to the cover piece above the pump and the other end to the next cover piece below the pump.

From Pump to Roller Clamp

One 7″×12″ fabric piece

Two 2″ pieces of ¼″ elastic

3 fasteners

Turn side edges over ½″ and finish. Sew ½″ casing at top and bottom edges. Insert 2″ pieces of elastic into casings and sew each end. Place fasteners about 5 ½″ apart. Pump strap above should be attached to the top edge and roller clamp strap below should be attached to the bottom edge.

Roller Clamp Strap

One 1 ½″×4″ fabric piece

One 3″ piece of 14″ elastic

Fold right side in, sew ⅛″ lengthwise seam. Turn right side out. Insert elastic and sew both ends. Sew one end to the cover piece above and the other end to the end of line cover piece below.

From Roller Clamp to End of Line

One 7″×78″ fabric piece (add 1″ to total if two pieces needed to make 78″, sew seam between pieces)

Two 2″ pieces of ¼″ elastic

18 fasteners

Turn over side edges ½″ and finish. Sew ½″ casing at top and bottom edges. Insert 2″ pieces of elastic into casings and sew each end. Place fasteners about 5 ½″ apart. Place two extra fasteners between 23″; and 26 ½″ from top edge for syringe pump line to be connected to IV line.

Pump Strap for syringe pump cover

One 1 ½″×10 ½″ fabric piece

One 9″ piece of ¼″ elastic

Fold right side in, sew ⅛″ lengthwise seam. Turn right side out. Insert elastic and sew both ends. Sew one end to back edge of syringe pump cover and sew a fastener on the front edge of strap and top front edge over syringe pump line cover.

Syringe Pump Line Cover

One 7″×37″ fabric piece

9 fasteners

Fold fabric in half lengthwise and cut an angle from 34 ½″ on the open edge to 37″ on the folded edge, turn over side edges ½″ and finish. Place fasteners about every 5 ⅕″ apart and two fasteners on angle edge.

The intravenous tubing line covers are preferably constructed from flannel, polyester, or cotton-blend material. However, any opaque fabric is suitable. Soft fabrics are preferred in that they are non-irritating to the skin and have been found to “soften” the nature of the intravenous fluid infusion and medicine infusion from the perspective of the parents of neonatal patients or pediatric patients. These fabrics can be in nursery style colors with any desired patterns such as cartoon characters, any other animated pictures, or any pattern suitable for children, on the fabrics.

The IV tubing covers as described herein accomplish the primary objective of providing a reusable and thus economic shielding mechanism for use in the delivery by intravenous infusion of light-sensitive fluids and medications.

1. An apparatus for dispensing light sensitive fluids to a patient, comprising:
a pump mechanism for pumping the fluid;
a first tubing connected to said pump mechanism to deliver the fluids pumped to the patient; and
a first opaque fabric member covering over said tubing to block the light accessible to the light sensitive fluid.

2. The apparatus of claim 1, wherein said pump mechanism is a syringe pump.

3. The apparatus of claim 1, wherein said pump mechanism is an infusion control pump.

4. The apparatus of claim 3, further comprising:
an intravenous (IV) bag interconnected to said infusion control pump;
a second tubing connected between said IV bag and said infusion control pump; and
a second opaque fabric member covering said second tubing.

5. The apparatus of claim 4, further comprising an opaque fabric bag member covering said IV bag.

6. The apparatus of claim 4, further comprising a strap interconnecting said first and second opaque fabric members.

7. The apparatus of claim 4, wherein said first and second opaque fabric members are elongate and tubular in configuration of sufficient diameter and length to substantially cover said first tubing.

8. The apparatus of claim 4, further comprising:
a syringe pump;
a third tubing interconnected between said syringe pump and said first tubing; and
a third opaque fabric member covering said third tubing.

9. The apparatus of claim 8, wherein said first opaque fabric member includes an opening therethough for the entry of said third tubing.

10. The apparatus of claim 9, wherein said first and third opaque fabric members have connection members to hold them together.

11. The apparatus of claim 1, wherein said first opaque fabric member is elongate and tubular in configuration of sufficient diameter to cover said first tubing.

12. A method of dispensing light sensitive fluids to a patient, comprising the steps of:
pumping the fluid though a first tubing connected to said pump to deliver the fluids pumped to the patient; and
covering said first tubing with a first opaque fabric member to block the light accessible to the light sensitive fluid.

13. The method of claim 12, wherein said fluid is pumped with a syringe pump.

14. The method of claim 12, wherein said fluid is pumped with an infusion control pump.

15. The method of claim 13, further comprising the steps of:
interconnecting an intravenous (IV) bag to said infusion control pump;
connecting a second tubing between said IV bag and said infusion control pump;
covering said second tubing with a second opaque fabric member; and
covering said intravenous bag with an opaque bag cover..

16. The method of claim 15, further comprising the step of interconnecting said first and second opaque fabric members with a strap.

17. The method of claim 15, wherein said first and second opaque fabric members are provided as elongate and tubular in configuration, and of sufficient diameter and length to substantially cover said first and second tubing.

18. The method of claim 15, further comprising the steps of:
providing a syringe pump;
interconnecting a third tubing between said syringe pump and said first tubing; and
covering said third tubing with a third opaque fabric member.

19. The method of claim 18, wherein said first opaque fabric member is provided with an opening therethough for the entry of said third tubing.

20. The method of claim 19, wherein said first and third opaque fabric members are held together by connection members.
